(19) United States
(12) Patent Application Publication
(10) Pub. No.: US 2005/0133038 A1
(43) Pub. Date: Jun. 23, 2005

(54) Adjustable collar and retainer for endotracheal tube

(76) Inventor: Michael John Rutter, Cincinnati, OH (US)

Correspondence Address: HASSE GUTTAG & NESBITT LLC, 7550 CENTRAL PARK BLVD.,, MASON, OH, 45040, US

(21) Appl. No.: 10/740,255

(22) Filed: Dec. 18, 2003

(51) Int. Cl. A62B 9/06; A61M 16/00

(52) U.S. Cl. 128/207.17

(57) ABSTRACT

An adjustable collar and retainer for a nasal or oral endotracheal tube are disclosed. The collar comprises a smooth first surface for contacting the skin of the patient, a support section attached to the first surface, and a band attached to the support section comprising flexible domes that compress against the tube and grip it when the collar is secured around the tube. The retainer comprises the adjustable collar and lateral extensions from the collar for securing the collar to the head of the patient. The retainer optionally further comprises at least one strap attached to the lateral extensions for securing the collar to the head of the patient.

### BACKGROUND OF THE INVENTION

This invention relates to an adjustable collar for a nasal or oral endotracheal tube, and a retainer comprising the collar useful for securing the nasal or oral endotracheal tube in a patient. More particularly, the endotracheal tube retainer comprises the adjustable collar and lateral extensions from the collar useful for securing the collar to the head of the patient. The retainer optionally further comprises at least one strap attached to the lateral extensions for securing the collar to the head of the patient.

During intubation, an endotracheal tube is inserted into the patient's trachea through the nasal or oral cavity. Patients in intensive care units frequently require prolonged intubation. Prolonged intubation, whether nasally or orally, may have associated endotracheal tube related consequences. The most important of these is inadvertent extubation or alteration of tube position. Additional problems may be seen with pressure ulceration of the nostril or skin reactions where the endotracheal tube is secured to the patient's skin. Traditionally, endotracheal tubes have been held in position with tape or cloth ties. These are prone to the collection of secretions (with associated infection), are difficult to change, and tend to loose their ability to secure the tube with time. They are also problematic for adult males with facial hair.

U.S. Pat. No. 5,934,276, Fabro, et al., discloses an endotracheal oral tube holder containing a face anchor and a tube cradle. The face anchor has buckles that hold a head harness. U.S. Pat. No. 5,076,269, Austin, discloses an endotracheal tube retaining apparatus that includes a soft, flexible plate for engaging against the patient's upper lip, with a ring fastener for connecting the soft plate to the endotracheal tube. U.S. Pat. No. 6,408,850, Sudge, discloses a medical tube holder that fastens around a medical tube through interlocking ends, and means for attaching a harness assembly.

Despite these and other devices known in the art, there is a continuing need for a device that can securely retain an endotracheal tube in position for a period of time, is hygienic and protective of surrounding tissues, and can easily be adjusted or removed.

### BRIEF DESCRIPTION OF THE INVENTION

The present invention relates to an adjustable collar for a nasal or oral endotracheal tube, said collar comprising:
a) a smooth first surface for contacting the skin of a patient using the endotracheal tube,
b) a support section attached to the first surface, and
c) a band attached to the support section comprising flexible domes that compress against the tube and grip it when the collar is secured around the tube.

The invention also relates to a retainer for securing a nasal or oral endotracheal tube in a patient, said retainer comprising:
a) an adjustable collar comprising:
   (i) a smooth first surface for contacting the skin of the patient,
   (ii) a support section attached to the first surface, and
   (iii) a band attached to the support section comprising flexible domes that compress against the tube and grip it when the collar is secured around the tube, and
b) lateral extensions from the collar for securing the collar to the head of the patient.

In another aspect, the invention relates to a retainer as described above further comprising at least one strap attached to the lateral extensions for securing the collar to the head of the patient.

### BRIEF DESCRIPTION OF THE DRAWINGS

FIG. 1 is a frontal view of an endotracheal tube retainer of the invention securing a nasal endotracheal tube in a patient.

FIG. 2 is a side view of the retainer of FIG. 1 secured to the head of the patient, with the nasal endotracheal tube held in place.

FIG. 3 is an enlarged partial side view of the adjustable collar component of the retainer of FIG. 1, with the collar being closed around the nasal endotracheal tube and securing it in position.

FIG. 4 is a plan view of a retainer of the invention, with the adjustable collar being in the open or non-engaged position.

FIG. 5 is an enlarged view of the adjustable collar of FIG. 4, with the collar being in a partially closed position around the endotracheal tube.

FIG. 6 is an enlarged view of the adjustable collar of FIG. 4, with the collar being in a closed position around the endotracheal tube.

FIG. 7 is a frontal view of an endotracheal tube retainer of the invention securing an oral endotracheal tube in a patient.

FIG. 8 is an enlarged view of the adjustable collar component of the retainer of FIG. 7, with the collar being in the open or non-engaged position.

FIG. 9 is an enlarged view of the adjustable collar component of FIG. 8, with the collar being in a partially closed position around the endotracheal tube.

### DETAILED DESCRIPTION OF THE INVENTION

The present invention relates to an adjustable collar for a nasal or oral endotracheal tube. The adjustable collar comprises a) a smooth first surface for contacting the skin of a patient using the endotracheal tube, b) a support section attached to the first surface, and c) a band attached to the support section comprising flexible domes that compress against the tube and grip it when the collar is secured around the tube. The invention also relates to a retainer for securing a nasal or oral endotracheal tube in a patient, the retainer comprising the above adjustable collar and lateral extensions from the collar for securing the collar to the head of the patient. The retainer typically further comprises at least one strap attached to the lateral extensions for securing the collar to the head of the patient.

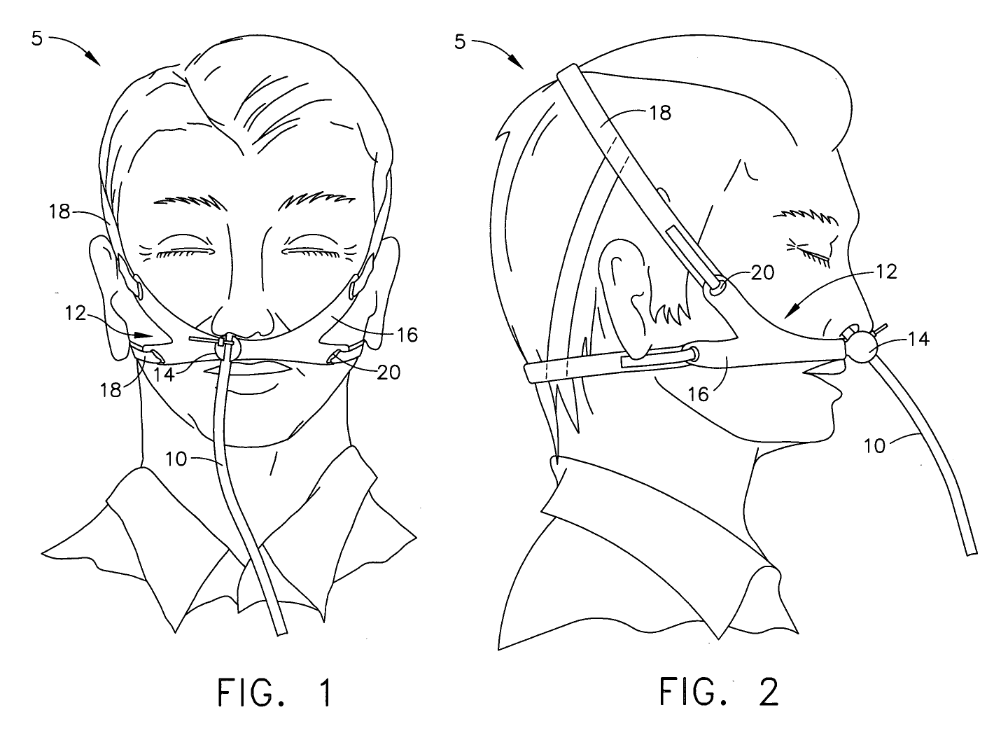

In the embodiment shown in FIG. 1, patient 5 is intubated with an endotracheal tube 10 secured in the nasal cavity by endotracheal tube retainer 12. Retainer 12 comprises an adjustable collar 14 that surrounds and grips tube 10 and holds it in place in the patient's nasal cavity.

Adjustable collar 14 is made of a flexible, non-irritating plastic material that is ergonomically configured to comfortably fit the patient's face during use. Collar 14 is designed to have sufficient strength and stability to hold endotracheal tube 10 in place during use, including when equipment such as a ventilator or monitor is attached to the distal end of the tube. When used with a nasal endotracheal tube, collar 14 conforms to the shape and size of the patient's face in the area beneath the nose and above the upper lip of the patient. The collar is also designed to flex with movement in the contact area of the patient's skin. The collar retains its shape during use, but has sufficient “give” to relieve pressure on the patient's face. The width of the collar is selected to dissipate the pressure that occurs when it is secured to the patient's face, thus preventing pressure sores. Collar 14 is typically made of silicone, polypropylene, polyethylene, polyvinylchloride, or similar material. In one embodiment, the collar is made of silicone, available as Dow Coming Silastic, medical grade.

Endotracheal tube retainer 12 also comprises lateral extensions 16 from adjustable collar 14 for securing the collar to the head of the patient. Lateral extensions 16 typically lie against the skin of the face, and thus have a smooth surface with no sharp edges that contact the skin. These lateral extensions are made of a flexible, non-irritating plastic material, often the same material used to form the adjustable collar 14. The lateral extension 16 and collar 14 are typically formed as one unit, although the collar can be a separate component that is attached to the lateral extensions, for example by snapping, clipping or hooking it in place.

Lateral extensions 16 may extend around the back of the patient's head and/or neck and secure adjustable collar 14 to the head of the patient. Typically however, retainer 12 further comprises at least one strap 18 attached to the lateral extensions that is used to secure the collar to the patient's head. In the embodiment shown in FIGS. 1 and 2, lateral extensions 16 have a bi-lobed or Y-shaped appearance to allow for attachment of two straps through eyelets 20. Various means may be used to attach the one or more straps to the lateral extensions, including hook and loop mechanical fasteners, snaps, buckles, buttons, ties, and the like.

In the embodiment shown in FIGS. 1 and 2, a lower strap 18 loops around the neck below the ears and an upper strap 18 loops around the head above the ears. In one embodiment, the straps comprise a hook and loop refastentable material, such as the VELCRO brand material, to allow for easy length alteration (as seen with tracheotomy ties). Such straps should lie laterally against the scalp skin so as not to damage the skin. As shown in FIG. 2, the straps can be connected to each other by at least one additional strap, e.g., two straps that attach vertically behind each ear. This minimizes the risk of the straps coming loose (e.g., to prevent the superior strap flipping up over the head).

In another embodiment, the lateral extensions are attached to a head harness comprising straps made of cloth, elastic plastic or rubber material adapted to fit the head of the patient and hold the retainer in place with little pressure on the face. The straps may go through self-locking buckles so that the device has multiple adjustable points. The straps typically have an upward pull so as to keep the device from slipping on the face.

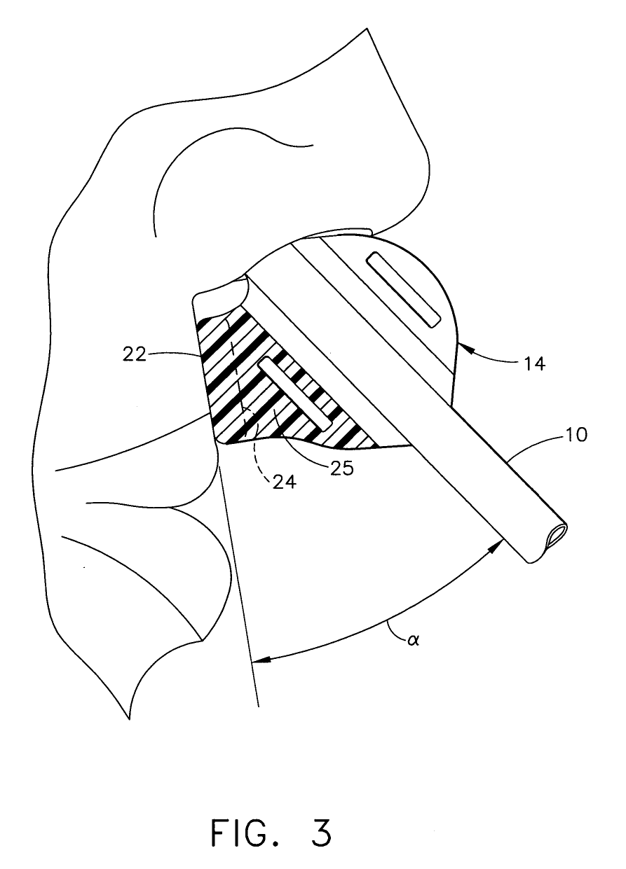

In the embodiment shown in FIG. 3, adjustable collar 14 comprises a smooth first surface 22 that contacts the patient's skin, in this case, in the region between the nose and the mouth. First surface 22 may be an element of a separate collar, such as shown in FIG. 3, or an element of a one-piece retainer comprising the collar and lateral extensions, such as shown in FIGS. 1-2. Alternatively, the collar may have a first surface that attaches to lateral extensions that directly contact the patient's skin. In either case, the first surface 22 should be smooth with no sharp angles where it contacts the skin, directly or indirectly. The first surface is made of a flexible plastic material that is non-irritating to the patient's skin, such as silicone, polypropylene or other conformable plastic that generally contours to the shape of the patient's face and has the ability to yield slightly to relieve any pressure on the face.

As shown in FIG. 3, adjustable collar 14 typically tapers in the region where it contacts tube 10 to allow for increased lateral movement of the edges of the collar and tube during use. This minimizes the risk of the tube kinking where it exits collar 14, and minimizes the risk of damage to the patient's skin through rubbing along first surface 22. When used with a nasal endotracheal tube, the adjustable collar typically holds the tube at an angle of from about 40° to about 70°, more typically from about 50° to about 60°, relative to the skin between the patient's nose and upper lip, whereby the tube is closer to the skin proximally than distally. This is shown as the angle alpha in FIG. 3. Such a configuration helps to protect the nostril skin while minimizing the bend on the tube as it exits the nostril. The angle alpha will vary dependent on the age of the patient and the diameter of the endotracheal tube.

Adjustable collar 14 further comprises a support section 25 that is connected to first surface 22. Support section 25 supports endotracheal tube 10 and helps hold it in place at the desired distance and angle relative to the patient's face. Support section 25 is located between first surface 22 and that portion of the adjustable collar that grips the endotrachael tube when the collar is secured around the tube. Support section 25 typically is connected to, e.g., it sits on top of or adjacent to, a second surface 24 of adjustable collar 14. Second surface 24 is generally parallel to first surface 22, and typically is the surface opposite first surface 22.

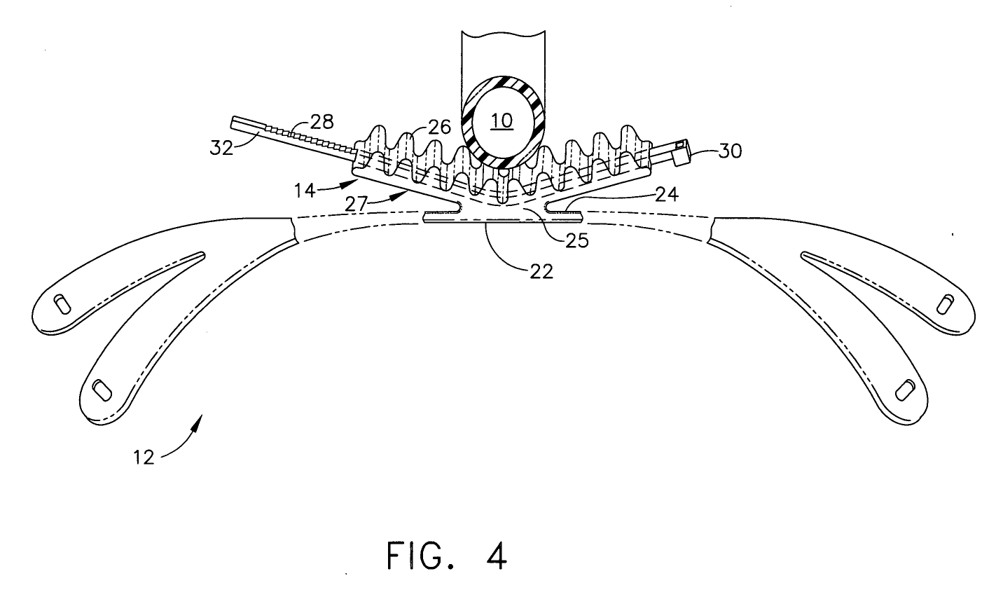

In the embodiment shown in FIG. 4, adjustable collar 14 of retainer 12 is shown in an open or non-engaged position. When attaching the collar to endotracheal tube 10, the collar initially lies flat and the tube is placed on it. Adjustable collar 14 comprises a smooth first surface 22 for contacting the skin of the patient, and a second surface 24, generally parallel to first surface 22. Support section 25 is attached to first surface 22 and second surface 24, e.g., it sits on top of second surface 24 and first surface 22 when adjustable collar 14 is viewed as shown in FIGS. 4-6.

Adjustable collar 14 further comprises a band 27 attached to support section 25 that comprises flexible domes 26 e.g., silicone domes, that compress against and grip endotracheal tube 10 when the collar is secured around the tube. The flexible domes have a high friction coefficient where they contact the tube to hold the tube in place in the patient. The domes may be in the form of dimples that have a circular base, or laterally extending ridges that have a domed surface which contacts the tube, as shown in FIG. 4. The domes typically have a height above the surface of from about 0.3 to about 3 mm, more typically from about 0.5 to about 2 mm. The domes may also have a flat or flattened top prior to contacting the tube. The domes are typically distributed substantially uniformly along the length of band 27 where they contact tube 10 when adjustable collar 14 is secured around the tube, but any distribution that allows the adjustable collar to grip the tube may be used.

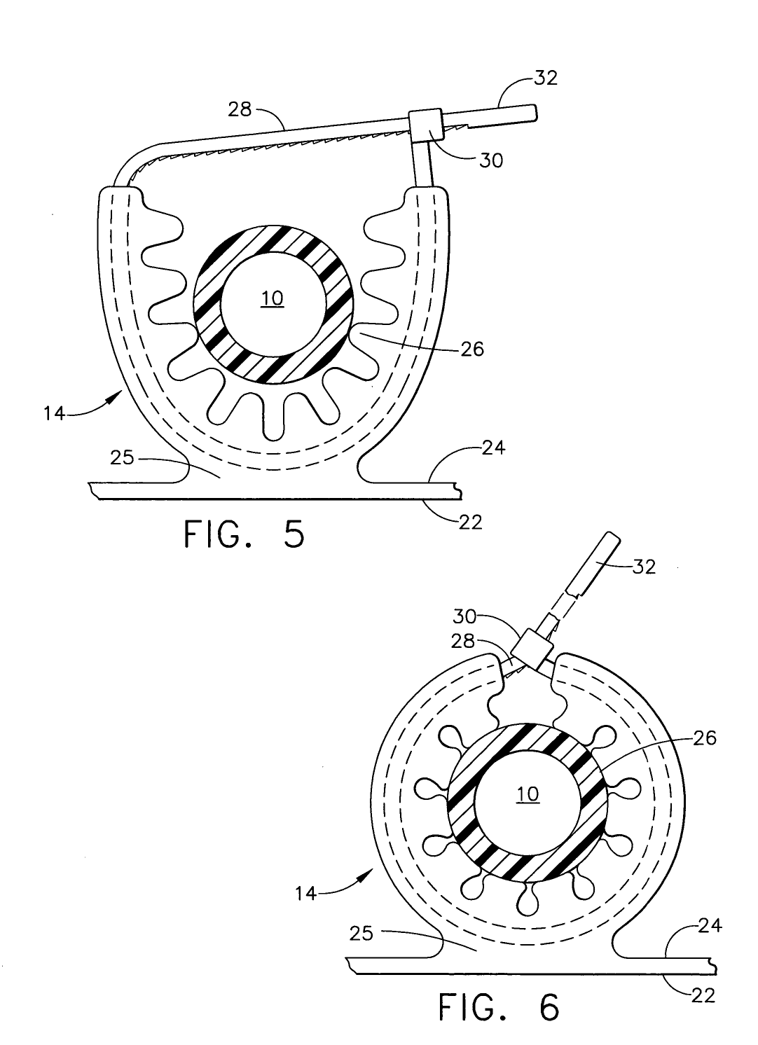

As shown in FIG. 4, band 27 typically also comprises a high strength strip of plastic, such as retainer strip 28, which typically underlies the domes. One end of the retainer strip 28 has a lock, such as ratcheted lock 30, while the other end has a belt, such as tapered belt 32. As shown in FIG. 5, belt 32 can be passed around endotracheal tube 10 and through lock 30. Retainer strip 28 is then partially tightened around tube 10 so that at least some of domes 26 contact the tube. The tube is held in place, but there is still sufficient space for some adjustment of the tube position. Once the desired tube position is achieved, belt 32 is pulled tight to secure collar 14 around tube 10, as shown in FIG. 6. In this position, domes 26 will be pulled tight against tube 10, without significantly compressing it and restricting air flow. The protruding end of belt 32 is typically cut off. A small gap between lock 30 and tube 10 allows one to easily cut retainer strip 28 and open adjustable collar 14 when tube 10 is no longer needed or the collar needs to be replaced. Other means may be used to secure the collar around the tube, such as the harness tie disclosed in U.S. Pat. No. 5,076,269 or the clamp and wrap around strap disclosed in U.S. Pat. No. 4,516,293. Alternatively, the ends of the collar may comprise snaps, buckles, or ties that can be used to secure the collar around the tube.

While the above embodiments have been described in the context of a retainer for a nasal endotracheal tube, the present invention also relates to a retainer for an oral endotracheal tube, and an adjustable collar for use therein.

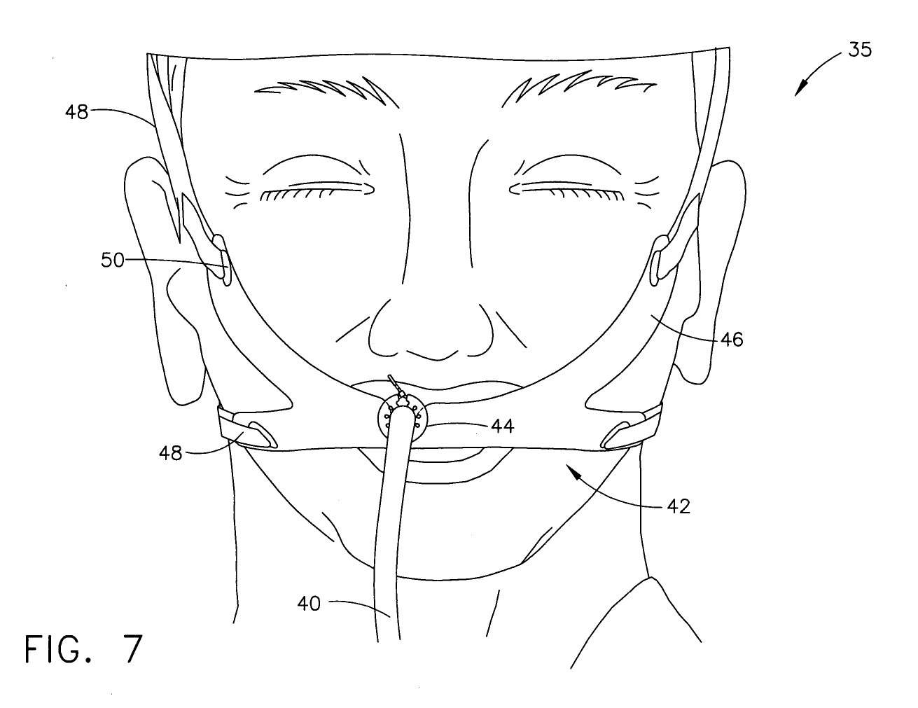

In the embodiment shown in FIG. 7, patient 35 is intubated with an endotracheal tube 40 secured in the oral cavity by endotracheal tube retainer 42. Retainer 42 comprises an adjustable collar 44 that surrounds and grips tube 40 and holds it in place in the patient's oral cavity.

Adjustable collar 44 is made of a flexible, non-irritating plastic material that is ergonomically configured to comfortably fit the patient's face during use. Collar 44 is designed to have sufficient strength and stability to hold endotracheal tube 40 in place during use, including when equipment such as a ventilator or monitor is attached to the distal end of the tube. Collar 44 is also designed to flex with movement in the contact area of the patient's skin, including the lips. The collar retains its shape during use, but has sufficient “give” to relieve pressure on the patient's face. The width of the collar is selected to dissipate the pressure that occurs when it is secured to the patient's mouth, thus preventing pressure sores. Collar 44 is typically made of silicone, polypropylene, polyethylene, polyvinylchloride, or similar material. In one embodiment, the collar is made of silicone, available as Dow Coming Silastic, medical grade.

Endotracheal tube retainer 42 also comprises lateral extensions 46 from adjustable collar 44 for securing the collar to the head of the patient. Lateral extensions 46 typically lie against the skin of the face, and thus have a smooth surface with no sharp edges that contact the skin. These lateral extensions are made of a flexible, non-irritating plastic material, often the same material used to form the adjustable collar 44. The lateral extension 46 and collar 44 are typically formed as one unit, although the collar can be a separate component that is attached to the lateral extensions, for example by snapping, clipping or hooking it in place.

Lateral extensions 46 may extend around the back of the patient's head and/or neck and secure adjustable collar 44 to the head of the patient. Typically however, retainer 42 further comprises at least one strap 48 attached to the lateral extensions that is used to secure the collar to the patient's head. In the embodiment shown in FIG. 7, lateral extensions 46 have a bi-lobed or Y-shaped appearance to allow for attachment of two straps through eyelets 50. Various means may be used to attach the one or more straps to the lateral extensions, including hook and loop mechanical fasteners, snaps, buckles, buttons, ties, and the like.

In the embodiment shown in FIG. 7, a lower strap 48 loops around the neck below the ears and an upper strap 48 loops around the head above the ears. In one embodiment, the straps comprise a hook and loop refastentable material, such as the VELCRO brand material, to allow for easy length alteration (as seen with tracheotomy ties). Such straps should lie laterally against the scalp skin so as not to damage the skin. The straps can be connected to each other by at least one additional strap, e.g., two straps that attach vertically behind each ear. This minimizes the risk of the straps coming loose (e.g., to prevent the superior strap flipping up over the head).

In another embodiment, the lateral extensions are attached to a head harness comprising straps made of cloth, elastic plastic or rubber material adapted to fit the head of the patient and hold the retainer in place with little pressure on the face. The straps may go through self-locking buckles so that the device has multiple adjustable points. The straps typically have an upward pull so as to keep the device from slipping on the face.

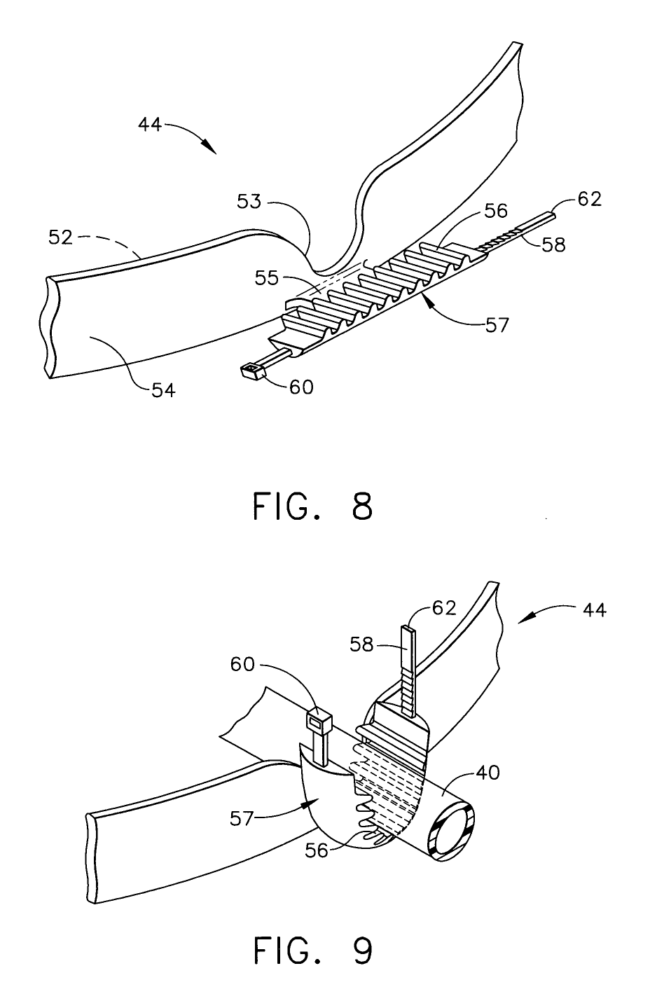

In the embodiment shown in FIG. 8, adjustable collar 44 comprises a smooth first surface 52 that contacts the patient's lips or surrounding skin. First surface 52 may be an element of a separate collar, or an element of a one-piece retainer comprising the collar and lateral extensions, such as shown in FIG. 7. Alternatively, the collar may have a first surface that attaches to lateral extensions that directly contact the patient's skin. In either case, the first surface should be smooth with no sharp angles where it contacts the skin, directly or indirectly. The first surface is made of a flexible plastic material that is non-irritating to the patient's skin, such as silicone, polypropylene or other conformable plastic that generally contours to the shape of the patient's face and has the ability to yield slightly to relieve any pressure on the face.

Adjustable collar 44 further comprises a support section 55 that is connected to first surface 52. Support section 55 supports endotracheal tube 40 and helps hold it in place at the desired distance and angle relative to the patient's face. Support section 55 is located between first surface 52 and that portion of the adjustable collar that grips the endotrachael tube when the collar is secured around the tube. Support section 55 typically is connected to, e.g., it sits on top of or adjacent to, a second surface 54 of adjustable collar 44. Second surface 54 is generally parallel to first surface 52, and typically is the surface opposite first surface 52.

In the embodiment shown in FIG. 8, adjustable collar 44 is shown in an open or non-engaged position. When attaching the collar to the endotracheal tube, the collar initially lies flat and the tube is placed on it though an open section, such as cradle 53, in the top portion of the collar. Adjustable collar 44 comprises a smooth first surface 52 for contacting the skin of the patient, and a second surface 54, generally parallel to first surface 52. Support section 55 is attached to first surface 52 and second surface 54, e.g., it sits adjacent to second surface 54 and first surface 52 when adjustable collar 44 is viewed as shown in FIGS. 8-9.

Adjustable collar 44 also comprises band 57 attached to support section 55 comprising flexible domes 56, e.g., silicone domes, that compress against endotrachael tube 40 and grip it once the collar is secured around the tube. The flexible domes have a high friction coefficient where they contact the tube to hold the tube in place in the patient. The domes may be in the form of dimples that have a circular base, or laterally extending ridges having a domed surface which contacts the tube, as shown in FIG. 8. The domes typically have a height above the surface of from about 0.3 to about 3 mm, more typically from about 0.5 to about 2 mm. The domes may also have a flat or flattened top prior to contacting the tube. The domes are typically distributed substantially uniformly along the length of band 57 where they contact tube 40 when adjustable collar 44 is secured around the tube, but any distribution that allows the adjustable collar to grip the tube may be used.

As shown in FIGS. 8 and 9, band 57 typically also comprises a high strength strip of plastic, such as retainer strip 58, which typically underlies the domes. One end of the retainer strip 58 has a lock, such as ratcheted lock 60, while the other end has a belt, such as tapered belt 62. Belt 62 can be passed around endotracheal tube 40 and through lock 60. Retainer strip 58 is then partially tightened around tube 40 so that at least some of domes 56 contact the tube. The tube is held in place, but there is still sufficient space for some adjustment of the tube position. Once the desired tube position is achieved, belt 62 is pulled tight to secure collar 44 around tube 40. In this position, domes 56 will be pulled tight against tube 40, without significantly compressing it and restricting air flow. The protruding end of belt 62 is typically cut off. A small gap between lock 60 and tube 40 allows one to easily cut retainer strip 58 and open adjustable collar 44 when tube 40 is no longer needed or the collar needs to be replaced. Other means may be used to secure the collar around the tube, such as the harness tie disclosed in U.S. Pat. No. 5,076,269 or the clamp and wrap around strap disclosed in U.S. Pat. No. 4,516,293. Alternatively, the ends of the collar may comprise snaps, buckles, or ties that can be used to secure the collar around the tube.

The endotracheal tube retainer herein may vary in style and size, depending on the needs of the patient. For example, the retainer may be sized for various endotracheal tubes, which typically have an outside diameter ranging from 2.0 mm through 10.0 mm, in 0.5 mm increments.

Although various embodiments of the invention have been described and exemplified, it will be understood that the scope of the invention is not limited to that description. Changes and modifications will occur to those of ordinary skill in the art and they can be made without departing from the spirit and scope of the invention. The invention is considered to include the methods of accomplishing the results described herein as well as structures designed to accomplish them.

As used herein, the term “comprising” means various components, capabilities and/or steps can be conjointly employed in the present invention. Accordingly, the term “comprising” encompasses the more restrictive terms “consisting essentially of” and “consisting of”.

1. An adjustable collar for a nasal or oral endotracheal tube, said collar comprising:
a) a smooth first surface for contacting the skin of a patient using the endotracheal tube,
b) a support section attached to the first surface, and
c) a band attached to the support section comprising flexible domes, having a height above the surface of the band of from about 0.3 to about 3 mm, that compress against the tube and grip it when the collar is secured around the tube without significantly compressing the tube and restricting air flow.

2. The collar of claim 1 made of a semi-rigid, non-irritating plastic material.

3. The collar of claim 2 made of silicone.

4. The collar of claim 1 for use with a nasal endotracheal tube, wherein said collar is tapered in the region where it contacts the endotracheal tube.

5. The collar of claim 4 shaped so that it holds the endotracheal tube at an angle relative to the skin between the patient's nose and upper lip whereby the tube is closer to the skin proximally than it is distally.

6. The collar of claim 1 wherein the flexible domes are made of silicone.

7. The collar of claim 6 wherein a plastic retainer strip underlies the domes.

8. The collar of claim 7 wherein the plastic strip comprises a lock at one end and a belt at the other end that can be pulled to secure the collar around the tube.

9. A retainer for securing a nasal or oral endotracheal tube in a patient, said retainer comprising:
a) an adjustable collar comprising:
(i) a smooth first surface for contacting the skin of the patient,
(ii) a support section attached to the first surface, and
(iii) a band attached to the support section comprising flexible domes, having a height above the surface of the band of from about 0.3 to about 3 mm, that compress against the tube and grip it when the collar is secured around the tube without significantly compressing the tube and restricting air flow, and
b) lateral extensions from the collar for securing the collar to the head of the patient.

10. The retainer of claim 9 made of silicone.

11. The retainer of claim 9 wherein a plastic retainer strip underlies the domes.

12. The retainer of claim 9 wherein the lateral extensions have a bi-lobed appearance.

13. The retainer of claim 12 wherein the lateral extensions have a smooth surface that contacts the patient's skin.

14. A retainer for securing a nasal or oral endotracheal tube in a patient, said retainer comprising:
a) an adjustable collar comprising:
(i) a smooth first surface for contacting the skin of the patient,
(ii) a support section attached to the first surface, and
(iii) a band attached to the support section comprising flexible domes, having a height above the surface of the band of from about 0.3 to about 3 mm, that compress against the tube and grip it when the collar is secured around the tube without significantly compressing the tube and restricting air flow;
b) lateral extensions from the collar for securing the collar to the head of the patient; and
c) at least one strap attached to the lateral extensions for securing the collar to the head of the patient.

15. The retainer of claim 14 wherein the at least one strap comprises hook and loop refastenable material.

16. The retainer of claim 14 wherein at least one lateral strap connects the at least one strap attached to the lateral extensions.

17. The retainer of claim 14 made of silicone.

18. The retainer of claim 17 wherein the lateral extensions have a bi-lobed appearance.

19. The retainer of claim 18 wherein a plastic retainer strip underlies the domes.

20. The retainer of claim 19 wherein two straps are attached to the lateral extensions and two straps vertically connect the straps attached to the lateral extensions, and said straps comprise hook and loop refastentable material.
